(19) United States
(12) Patent Application Publication
(10) Pub. No.: US 2008/0269541 A1
(43) Pub. Date: Oct. 30, 2008

(54) Mechanized, adjustable magnetic device for therapy

(76) Inventor: David L. Stokesbary, Laguna Hills, CA (US)

Correspondence Address: FRANK C. PRICE, 13812 Sand-Hurst Pl, Santa Ana, CA, 92705, US

(21) Appl. No.: 11/818,976

(22) Filed: Jan. 24, 2007

(51) Int. Cl. A61N 2/06

(52) U.S. Cl. 600/9

(57) ABSTRACT

A magnetic therapy apparatus has adjustable space between the poles to accommodate various thicknesses of the body parts that are to be treated. This eliminates the inconvenience of exchanging various pole sizes for the various targets to be treated. The entire treatment zone can be rotated to accommodate both vertical and horizontal targets. The heavy apparatus includes a transportation dolly and a hydraulically actuated system for tilting the axis of the poles to various positions. An alternative system of varying the distances between the poles uses two rotating arms cantilevered with the poles at the extremities of the arms.

No federally sponsored research funds were used in the creation of the material of this application.

### BACKGROUND OF THE INVENTION

Devices for magnetic therapy normally are very heavy. Both the simple magnets used and the electromagnets that create the magnetic field are heavy. The usual machine has a place for the target body part to reside and a holder for the magnetic device. Magnets of different lengths are placed in a fixed holder frame in order to reach close proximity to a given body part situated in the machine. Multiple sizes of magnet are needed for flexibility to meet the needs of various body configurations. Usually, any size of therapeutic magnetic is heavy and cumbersome to handle by the machine operator.

This invention offers the convenience for an operator of magnets, permanently installed magnets, with adjustability so that a wide range of sizes of body parts can be accommodated at the push of a button that moves the magnets toward or away from a given body part surface. Also, the position of magnets can be changed to accommodate different postures of the human subject. Additional features of the machine to facilitate its moving and installation are a transport tractor assembly and telescoping cylinders to enable the tilting of the machine to its more vertical position for operation after being transported to a given site on transport wheels. These features are new to the business of using heavy, large magnets for therapy.

### SUMMARY OF THE INVENTION

The magnetic therapy machine consists of adjustable upper and fixed lower sources of magnetic fields that form, in effect, two magnetic poles. These can be coils or permanent magnets. The adjustment of a jack screw changes the distance between the sources that are the magnetic poles; the distance accommodates the thickness of the human body part that lies in between the poles. The adjustment of the upper pole can be made with a jackscrew. The screw can be driven by machine or by hand. The frame, or headstock, a “U” shape, can be positioned on its side or in a normal, upright “U” configuration or in any position in between. The change in position is accomplished with a telescoping hydraulic cylinder/actuator. The “U” frame serves as the return path of the formed magnetic field.

The apparatus has a removable transport tractor assembly that is a convenience for moving such a heavy device. A single unit can accommodate a person in a sitting or a prone position. A double unit, side-by-side, can be used for wider treatment of a whole human body in prone position. In another version of varying the distance between poles, the change is made by having the two poles mounted on the ends of arms with the opposite ends of the arms hinged.

### BRIEF DESCRIPTION OF THE DRAWINGS

FIG. 1 shows a front view of one form of the adjustable device—adjustable vertical to horizontal

FIG. 2 shows a side view of the unit in FIG. 1 with designated part numbers.

FIG. 3 shows a front view of a double unit, two units joined together using pole pieces.

FIG. 4 shows infinite radial positioning of the magnet headstock, actuated by a linear actuator.

FIG. 5 shows magnet rotational capability from vertical to horizontal for the magnet pair, the air gap adjustment cylinder being absent to expose the front view of the magnet assembly.

FIG. 6 shows a side view of the permanent magnet system and its key components.

FIG. 7 shows an end view of the permanent magnet housing with the attachment feature for various pole pieces.

FIG. 8 shows a rear view of the magnet rotator assembly cabinet with various rotational lock arm positions.

FIG. 9 shows a sectional view exposing the hydraulic cylinder.

FIG. 10 shows an average sized person sitting between the magnetic poles.

FIG. 11 shows an obese person in a supine position between the magnetic poles.

FIG. 12 shows a thin person as in FIG. 11.

FIG. 13 shows a thin person in supine position between pole pieces attached to two magnet systems.

### DETAILED DESCRIPTION

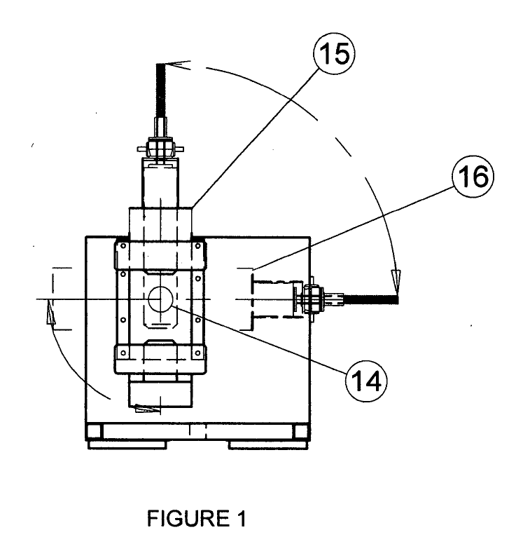

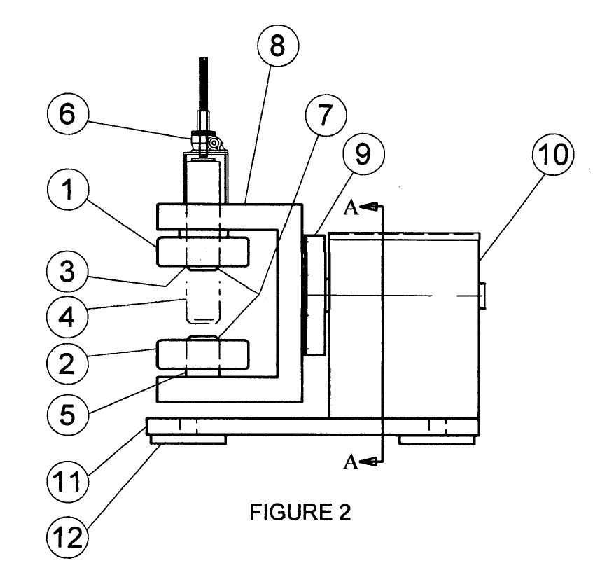

The system of magnet pairs in FIG. 1 can be rotated ninety degrees as indicated by the arcs and arrows. The shaft for rotation is 14. The dual magnet system 15 is shown in the vertical position. With the horizontal position 16 shown somewhat in phantom. FIG. 2 is a side view of the system in vertical position. The upper and lower magnetic coils are 1 and 2. The adjustable upper coil 3 is seen fully retracted and it's lower position is shown in phantom as 4. 5 is the fixed lower core. 6 is a screw jack enabling the core to be moved to adjust the air gap. 7 is a removable pole tip. Various configurations of pole tip can change the concentration of magnetic energy passing through the upper and lower cores. The “U” frame 8 completes the magnetic circuit induced by the coils or permanent magnets 2 and 1. The headstock 9 provides a mounting point for the magnets and their return path. The cabinet 10 is for equipment described later. The base 11 has compressed air casters 12 to enhance manual movement of the very heavy system.

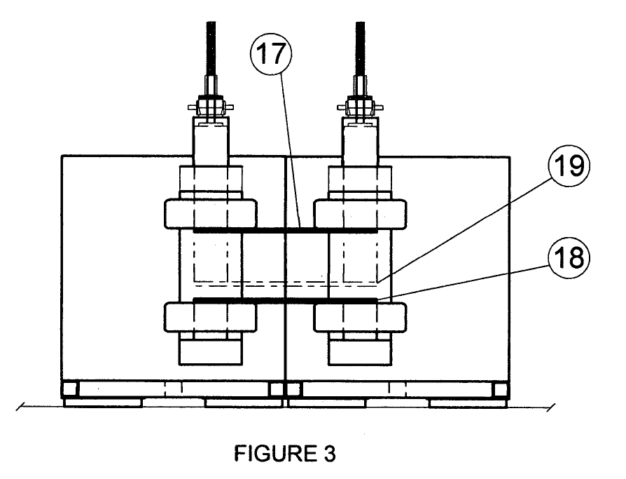

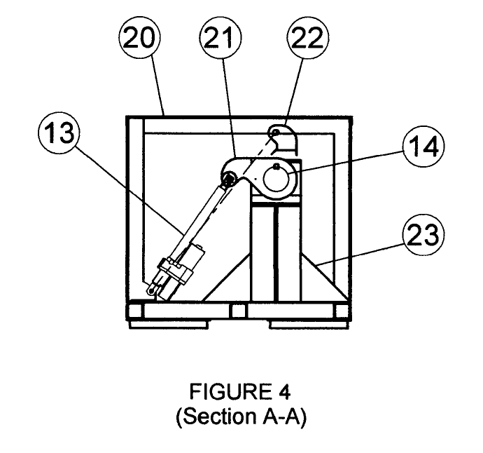

In FIG. 3 are seen two units joined together by 17 and 18, bar pole pieces connecting magnet cores. In phantom is seen the upper poles lowered to 19, obviously to reach a thinner body. FIG. 4 is a section view of the cabinet 10 showing the linear actuator 13. It provides infinite radial positioning of the magnet headstock. 13 is the linear actuator. 14 is the rotator shaft. 20 is the rotator equipment cabinet lid. 21 is the rotator shaft crank shown when the linear actuator is retracted and the magnet is in a horizontal position. 22 is the crank in position for the magnet to be in a vertical position; linear actuator 13 is extended. 23 is a shaft support brace.

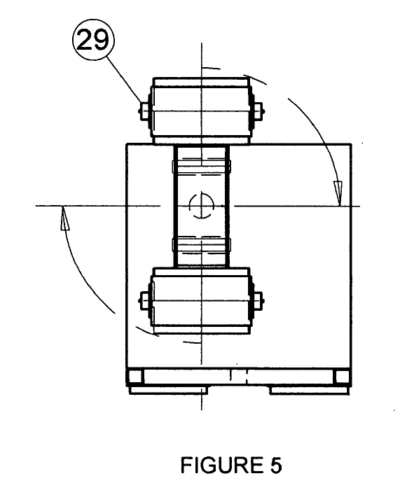

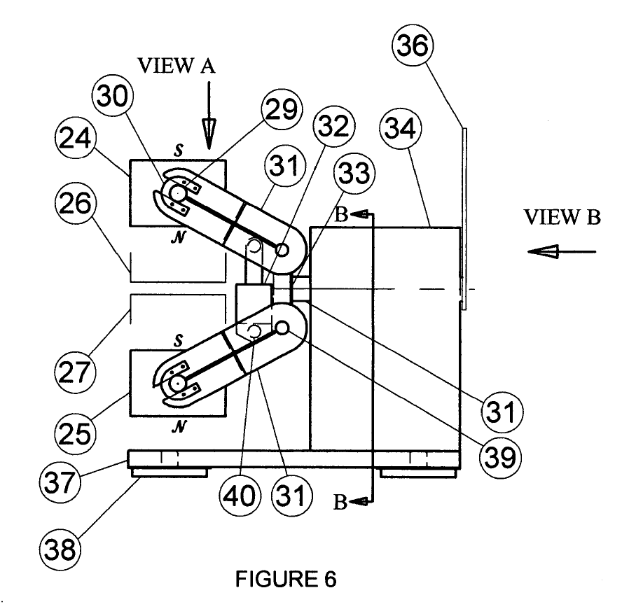

In FIG. 5, a front view of the permanent magnet system with arrows showing how the system can rotate. 29 is a self-aligning magnet pivot. In FIG. 6 can be seen 24 and 25 the magnets at maximum air gap, 26 and 27 at minimum gap. 29 is a self-aligning magnet pivot. Natural attraction of opposite poles causes the magnet faces to self-align. Swivel stops keep faces within alignment range. 30 is a magnet pivot pin retainer. Magnet housings can be can be slid into place and secured with these retainers. 31 is the air gap adjustment arm. It connects the magnet housing to the headstock and provides air gap adjustability. 32 is the air gap adjustment actuation cylinder. A non-metallic hydraulic cylinder provides infinite air gap adjustment. The cylinder is connected to each air gap adjustment arm 31. The headstock 33 provides a mounting point for the permanent magnet housings and adjustment linkage. 34 is the cabinet for the magnet rotator assembly—the same as item 10. 36 is the rotation lock arm that provides a method to lock the radial position of the magnet assembly. 37 is the base and 38 is an air caster. 39 is a pivot pin for the magnet air gap adjustment arm. It fastens the air gap adjustment arms to the headstock and allows radial movement when the cylinder is extended or retracted. 40 is the pivot pin for the air gap adjustment cylinder. The cylinder has pivot pins on both ends to attach the cylinder to the air gap adjustment arms 31 and accommodate the radial movement of the air gap adjustment arms when the cylinder is extended or retracted.

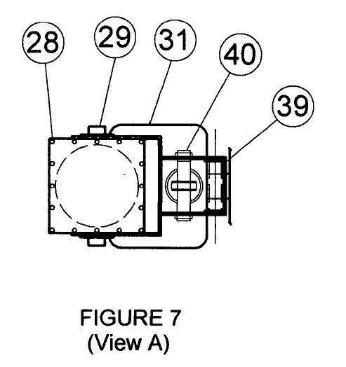

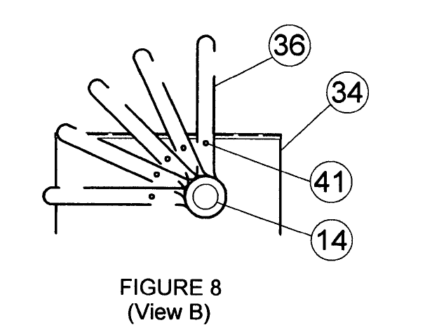

The end view of the permanent magnet housing, FIG. 7, shows the attachment feature for various pole pieces. Both ends of each permanent magnet housing are identical so that either end can be used to suit individual applications as necessary. 28 is the pole tip mounting bolt pattern. Each end of each magnet housing has a bolt pattern that provides a mounting location for unique pole pieces that are used to provide variations in magnetic flux patterns. 29 is a self-aligning magnet pivot such that magnet housings can be slid into place and secured with these retainers. 31 is the air gap adjustment arm that connects the magnet housing to the headstock and provides air gap adjustability. 39 is a pivot pin for the air gap adjustment cylinder that has pins on both ends to attach the cylinder to the air gap adjustment arms 31 and accommodate the radial movement of the air gap adjustment arms when the cylinder is extended or retracted. Pivot pin 40 is one of two on the air gap adjustment cylinder. On both ends, they attach the cylinder to the air gap adjustment arms 31 and accommodate the radial movement of the air gap adjustment arms when the cyliner is extended or retracted. In FIG. 8, a rear view of the magnet rotator assembly cabinet, can be seen various locking positions for the rotation lock arm 36. Again, 34 is the cabinet. 41 is the lock arm position lock pin that passes through the rotation lock arm 36 into one of the appropriate hole on the rotator assembly cabinet 34 and secures the radial positioning of the magnet assembly when the hydraulic power is shut off. 14 is the rotator shaft.

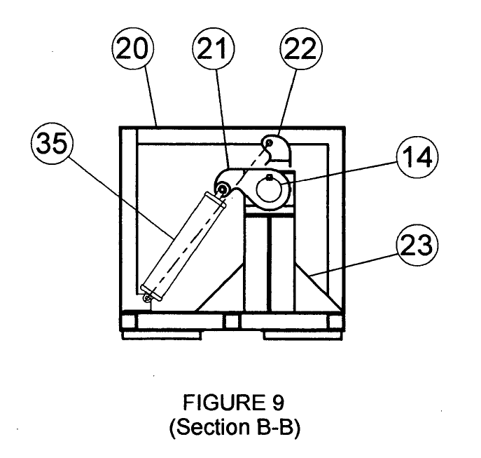

FIG. 9 is a different section through the cabinet. It shows the hydraulic cylinder in the same location as item 13, but requires a radial movement locking device such as the rotation lock arm 36 that is used on this system. 35 is the rotary positioning cylinder. It is constructed of non-magnetic metal because of its close proximity to the permanent magnetic housings. It provides radial positioning of the magnet assembly on the rotator shaft. Drives other than hydraulic could be used in this system.

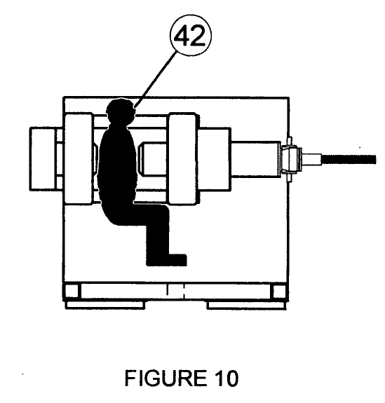

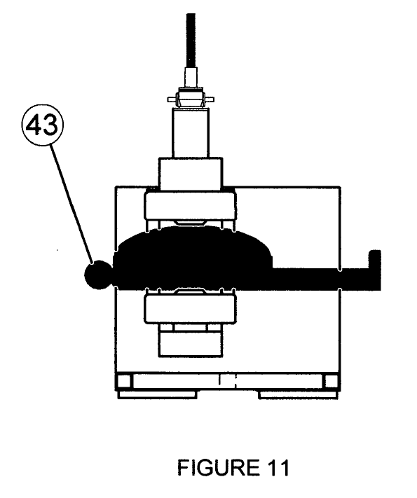

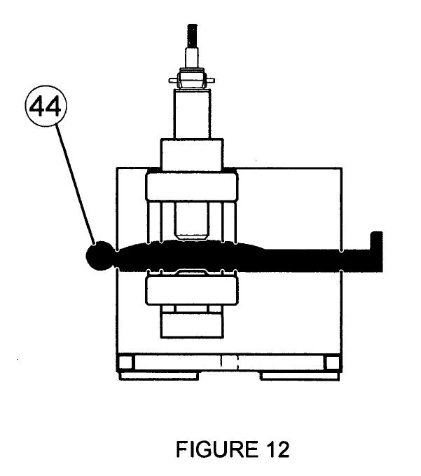

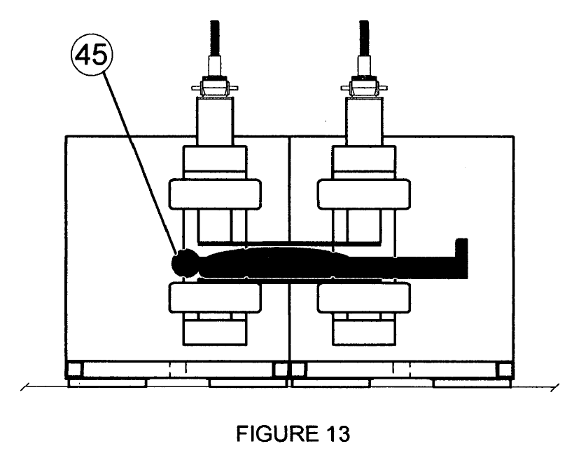

FIGS. 10, 11 and 12 shows how differently-sized people are accommodated by the machine in different positions. In FIG. 10 an average sized person is treated in a sitting position. In FIG. 11 an obese person is treated in a supine position. In FIGS. 12 and 13 and average size person is treated in supine position by one and by two magnetic systems.

I claim:

1. A system of one or more sets of magnets for use in therapy to treat persons by being arranged in close proximity to the various body parts of persons of various sizes and being situated in various positions, comprising:
containers of one or more north poles and one or more south poles, said containers equipped to move each said pole into various positions by actuation of hydraulic cylinders.

2. The system of claim 1 comprising a supporting framework and a floor-contacting base, said base resting on compressed air cushions that facilitate movement of the total system on flat, smooth floors.

3. The system of claim 1 comprising multiple magnet units arranged in tandem pairs of north and south poles to facilitate treatment simultaneously at more than one body location along a prone, outstretched body.

4. The system of claim 1 comprising rotational means to change the direction of the axis of each set of opposing magnetic poles from vertical to horizontal.

5. The system of claim 1 comprising means to change the distance between the poles to accommodate bodies and body parts of various thickness.
